(19) United States
(12) Patent Application Publication
(10) Pub. No.: US 2016/0169887 A1
(43) Pub. Date: Jun. 16, 2016

(54) SYSTEMS, DEVICES AND METHODS FOR A LATERAL FLOW ASSAY WITH SOLUTION ENHANCEMENT

(71) Applicant: Church & Dwight, Co., Inc., Princeton, NJ (US)

(72) Inventors: Albert R. Nazareth, Mercerville, NJ (US); Shang Li, Princeton Junction, NJ (US); Timothy Snowden, Howell, NJ (US); Giles H.W. Sanders, Hertfordshire (GB); Anthony Cass, Hertfordshire (GB)

(73) Assignee: Church & Dwight, Co., Inc., Princeton, NJ (US)

(21) Appl. No.: 14/570,973

(22) Filed: Dec. 15, 2014

(51) Int. Cl. G01N 33/561

(52) U.S. Cl. CPC G01N 33/561

(57) ABSTRACT

Systems, methods, devices and test kits for detecting an analyte and quantifying an analyte level in a biological fluid sample using a lateral flow assay with enhancement solution are disclosed. The fluid sample is applied to a lateral flow strip, and thereafter an enhancement solution is applied that enhances the results of the test. The enhancement solution is contained with the device, such as in a store or blister. The assay may also include a washing fluid applied to the strip in a similar manner.

### BACKGROUND

1. Field of the Invention

The present invention relates to diagnostic assays for detecting analytes in a liquid sample. In some embodiments, features for the detection of an analyte in a body fluid using a lateral flow assay with solution enhancement of the results are provided.

2. Description of the Related Art

Many types of ligand-receptor assays have been used to detect the presence of analytes in body fluids such as saliva, urine or blood. These assays typically involve antigen-antibody reactions, synthetic conjugates comprising enzymatic, fluorescent, or visually observable tags, and specially designed reactor chambers. In most of these assays, there is a receptor (e.g., an antibody) specific for the selected analyte (e.g., antigen), and a means for detecting the presence and/or amount of the antigen-antibody reaction product. More recent research has included the development of a number of artificial receptor systems such as imprinted polymers and oligonucleic and peptide aptamers. These systems have yet to find considerable use in commercial assays but may provide some advantages in niche cases such as when the analyte is a small molecule or where long term stability of the antibody is an issue. Although some commercially available tests are designed to make a quantitative determination, in many circumstances all that is required is a qualitative indication (e.g., positive/negative). Examples of such qualitative assays include blood typing, pregnancy testing, ovulation prediction and many types of urinalysis.

Diagnostic assays should generally be very sensitive because of the often low concentrations of analytes of interest present in a test fluid. False positives can be problematic, particularly with agglutination and other rapid detection methods such as dipstick and color change tests. Because of these problems, sandwich assays which use metal sols or other types of colored particles have been developed that rely on the interaction between avidin and biotin-tagged antibodies to provide enhanced sensitivity and specificity. For example, in some commercially available pregnancy tests, an antibody-antigen sandwich complex comprising a colloidal gold-labeled anti-hCG antibody and an anti-hCG biotin-labeled antibody is used. Test strips of this nature are known in the art, and are described in more detail in, for example, U.S. Pat. No. 6,319,676, the content of which is hereby incorporated by reference in its entirety.

Other proposals have sought to exploit the changes in electrical properties that occur due to the presence of metal-labeled antibodies. For example, International Application Publication No. WO2013/083686, the content of which is herein incorporated by reference in its entirety, discloses electrodes located on test and reference regions measuring changes in capacitance. A difference in capacitance between the test and reference regions is indicative of the presence and quantity of a bound metal and thus of the target analyte. However, in such systems the changes in the capacitance are small, and the layout thus requires precision. This requires more precise tools and other improvements to the designing, building and assembling of such devices. Thus, burdensome constraints are imposed on production, driving up costs for end users. Other approaches involve visual confirmation of results, such as viewing a color change in a test band. However, this presents problems with subjectivity in interpreting color changes that may be faint or otherwise difficult to see. In these contexts, there remains a need for further improvements in the design, reliability, and ease of manufacture for these and other assays and to provide the market with affordable test kits.

### SUMMARY

The embodiments disclosed herein each have several aspects no single one of which is solely responsible for the disclosure's desirable attributes. Without limiting the scope of this disclosure in any way, certain prominent features will now be briefly discussed, and such features may appear together or separately in one or more embodiments. After considering this discussion, and particularly after reading the section entitled “Detailed Description,” one will understand how the features of the embodiments described herein provide advantages over existing digital image capturing systems and methods.

In one aspect, a device for detecting an analyte in a fluid sample is disclosed. The device comprises a test strip and a first store. The test strip comprises a sample receiving region that receives the fluid sample and a capture region. The fluid sample flows laterally to the capture region upon receipt of the fluid sample by the sample receiving region. The first store is located upstream of the capture region, the first store containing a first enhancement solution and releasing the first enhancement solution onto the strip after the sample receiving region receives the fluid sample. Upon release onto the strip, the first enhancement solution flows laterally to the capture region.

In some embodiments, the device further comprises a second store located upstream of the capture region. The second store contains a second enhancement solution and releases the second enhancement solution onto the strip after the sample receiving region receives the fluid sample. Upon release onto the strip, the second enhancement solution flows laterally to the capture region.

In some embodiments, the first enhancement solution comprises a silver salt solution and the second enhancement solution comprises a hydroquinone initiator. In some embodiments, the first enhancement solution comprises chloroauric acid (HAuCl₄) and the second enhancement solution comprises Hydroxylamine hydrochloride (NH₂OH.HCl).

In some embodiments, the first store further comprises a release. Actuating the release allows the first enhancement solution to exit the first store and flow toward the strip.

In some embodiments, the device further comprises a wash store containing a washing fluid. The wash store releases the washing fluid onto the strip after the sample receiving region receives the fluid sample. Upon release onto the strip, the washing fluid flows laterally to the capture region. In some embodiments, the wash store further comprises a wash release, wherein actuating the wash release allows the washing fluid to exit the wash store and flow toward the strip.

In some embodiments, the release comprises a mechanical pusher configured to push the first enhancement solution from the at least one store and toward the strip. In some embodiments, the release comprises a puncture member configured to puncture the first store containing the first enhancement solution and thereby allow the first enhancement solution to exit the first store and flow toward the strip. In some embodiments, the release comprises at least one dissolvable port, wherein the strip is further configured such that, when the fluid sample is received by the sample receiving region, the fluid sample flows to the at least one port, wherein the at least one port is configured to dissolve after the fluid sample flows to the at least one port, and wherein dissolving the at least one port allows the first enhancement solution to be provided to the strip. In some embodiments, the device further comprises an electronic circuit comprising a processor operatively coupled to the release and/or the wash release. The processor is configured to execute a set of instructions to perform a method comprising actuating the release and/or the wash release.

In some embodiments, the device further comprises a plurality of electrodes at least partially aligned with the capture region and a processor. The processor is coupled to the electrodes and is configured to execute a set of instructions to perform a method comprising measuring an electrical property of the capture region. The release may be actuated after the measured electrical property of the capture region reaches a threshold value or a set time after the sample receiving region receives the fluid sample. In some embodiments, the measured electrical property is impedance and the threshold value is a threshold impedance.

In some embodiments, the first store comprises a first release and the second store comprises a second release. Actuating the first release causes the first enhancement solution to exit the first store and flow toward the strip, and actuating the second release causes the second enhancement solution to exit the second store and flow toward the strip. In some embodiments, the first and second release each comprise a mechanical pusher, a puncture member and/or a dissolvable port.

In some embodiments of the device, the strip further comprises a first antibody region comprising a first antibody that recognizes an epitope of the analyte, and a second antibody region comprising a second antibody that recognizes a different epitope of the analyte. In some embodiments, the first antibody is bound to a first label, and the second antibody is bound to a second label. The first antibody may be an anti-hCG antibody, and the second antibody may be an anti-hCG antibody. In some embodiments, the first label is one of gold, silver or polymer. In some embodiments, the second label is biotin.

In some embodiments, the capture region comprises an immobilized capture agent that captures the analyte. The immobilized capture agent may be monomeric or polymeric avidin. In some embodiments, the strip further comprises a release medium, a capture medium, an absorbent medium and a backing. The release medium may comprise the sample receiving region, a first antibody region comprising a first antibody that recognizes an epitope of the analyte, and a second antibody region comprising a second antibody that recognizes a different epitope of the analyte. In some embodiments, the capture medium comprises the capture region and a control region. In some embodiments, the strip further comprises a control region, wherein the strip is further configured such that when a fluid sample is applied to the sample receiving region, the fluid sample flows laterally to the control region, and wherein, upon release onto the strip, the first enhancement solution flows laterally to the control region.

In another aspect, a method for detecting an analyte in a fluid sample is disclosed. In some embodiments, the method comprises applying a fluid sample to a strip, wherein the strip is configured such that the fluid sample flows laterally to a capture region of the strip, and releasing a first enhancement solution from a first store coupled with the strip. The first enhancement solution flows from the first store and onto the strip after the sample receiving region receives the fluid sample. Upon flowing onto the strip, the first enhancement solution flows laterally to the capture region.

In some embodiments, the method further comprises releasing a second enhancement solution from a second store coupled with the strip. The second enhancement solution flows from the second store and onto the strip after the sample receiving region receives the fluid sample. Upon flowing onto the strip, the second enhancement solution flows laterally to the capture region. In some embodiments, releasing the first enhancement solution comprises actuating a first release to allow the first enhancement solution to flow from the first store. Actuating the first release may comprise mechanically pushing the first enhancement solution from the first store and toward the strip, or puncturing the first store containing the first enhancement solution to allow the first enhancement solution to exit the first store and flow toward the strip. In some embodiments, actuating the first release comprises flowing the fluid sample from the receiving region to a first dissolvable port coupled with the first store, and dissolving the first dissolvable port to allow the first enhancement solution to exit the first store and flow toward the strip.

In some embodiments, the method further comprises measuring an electrical property of the capture region. The first release may be actuated after the measured electrical property of the capture region reaches a threshold value. In some embodiments, the first release is actuated a set time after the sample receiving region receives the fluid sample. In some embodiments, releasing the first and second enhancement solutions comprises, respectively, actuating a first and second release to allow, respectively, the first and second enhancement solutions to flow from the first and second stores.

In some embodiments, the method further comprises releasing a washing fluid from a wash store and onto the strip after the strip receives the first and/or second enhancement solutions. Upon flowing onto the strip, the washing fluid flows laterally to the capture region. In some embodiments, releasing the washing fluid comprises actuating a wash release to allow the washing fluid to flow from the wash store. In some embodiments, the wash release is actuated a set time after the strip receives the first and second enhancement solutions.

In some embodiments, the measured electrical property is impedance and the threshold value is a threshold impedance.

### BRIEF DESCRIPTION OF THE DRAWINGS

FIGS. 1A-1C show different views of an embodiment of a lateral flow assay device with solution enhancement.

FIGS. 2A-2B show different views of a test strip, with stores containing enhancement solution(s), that may be located in the device of FIGS. 1A-1C.

FIGS. 3A-3B show perspective views of various release mediums with conductive areas that may be in the strip of FIGS. 2A-2B.

FIG. 3C shows a partial perspective view of a circuit board with electrodes that may be used with the release medium of FIG. 3B.

FIGS. 4A-4G show perspective views of various embodiments of electrode layouts about a measurement region on a membrane that may be part of the strip of FIGS. 2A-2B.

FIG. 5 shows a block diagram of an example electrical system that may be in the device of FIGS. 1A-1C.

FIG. 6 shows a circuit diagram of an embodiment of a circuit that may be utilized in the device of FIGS. 1A-1C.

FIG. 7A shows a flow chart of an embodiment of a method for using a lateral flow assay with enhancement solution(s).

FIGS. 7B-7C are flow charts illustrating methods of introducing enhancement solution(s), that may be used in the method of FIG. 7A.

FIGS. 7D-7F are flow charts illustrating methods for verifying that a strip is ready for introducing enhancement solution(s), that may be used in the method of FIG. 7C.

FIG. 7G is a flow chart illustrating a method for introducing solution(s) by actuation of a release, which may be used in the method of FIG. 7C.

FIGS. 7H-7J are flow charts illustrating methods for actuating various embodiments of releases, that may be used in the method of FIG. 7G.

FIG. 7K is a flow chart illustrating a method for introducing a washing fluid to the strip, that may be used in the method of FIG. 7A.

FIGS. 8A-8B show graphs displaying various results of impedance measurements for a capture region and a non-capture region from a lateral flow assay having solution enhancement.

FIGS. 9A-9B show schematics of a partial embodiment of a measurement region before and after, respectively, application of enhancement solution(s).

### DETAILED DESCRIPTION

The following detailed description is directed to certain specific embodiments of the development as described with reference to the accompanying figures. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Embodiments and examples of the invention will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention described herein.

The methods, devices, test kits and systems described herein are used to perform immunologically-based diagnostic tests. The devices described herein enable a user to determine with high accuracy and sensitivity the presence or absence of a biological marker which is indicative of a physiological condition or state. For example, the methods and devices described herein can enable untrained personnel to reliably assay a liquid sample for the presence of small quantities of a particular analyte, while avoiding false positives and simplifying test procedures. The devices described herein are ideal for use in over-the-counter test kits, which can enable a consumer to self-diagnose, for example, pregnancy, ovulation, venereal disease and other diseases, infections, or clinical abnormalities which result in the presence of an antigenic substance in a body fluid, including determination of the presence of metabolites of drugs or toxins. Some embodiments involve the use of a biphasic chromatographic substrate to achieve an easily readable, sensitive, reproducible indication of the presence of an analyte, such as human chorionic gonadotropin (hCG), follicle stimulating hormone (FSH), thyroid stimulating hormone (TSH) or luteinizing hormone (LH), in a test sample, such as a human urine sample. A variety of analytes can be detected in a variety of liquid samples, including urine, blood, saliva, or any other body fluid.

Disclosed herein are methods, systems and devices for providing solution enhancement in a lateral flow assay. Such enhancement may be provided through electroless deposition of gold, silver and/or other particles. The solution is used to enhance the signal to facilitate determining the results of an assay. For instance, solution may be applied to a visual-based lateral flow assay that will enhance the visual signal, such as the darkness or contrast of a colored test region. As another example, solution may be applied to an electrical property based lateral flow assay, such as an impedance or capacitance based assay, that enhances the electrical property signal measured across the test region. The solution may contain labels that enhance the results by depositing more of the label on the test region. For instance, the enhancement solution may contain metal particles that enhance the visual, electrical, or other signal at the test region that is indicative of the assay results. The solution may be contained in one or more stores with the device. The solution may be released from the stores after the test fluid sample has been applied. The stores may be self-contained with the device such that a user may easily and simply use the device without the need for complicated tools for applying the enhancement solution. The stores may have a release that allows the solution to flow from the store onto the strip. For example, the devices may have a mechanical pusher, a puncture member, a dissolvable port, or other releases that allow the solution to exit and flow from the stores. The assay may also contain a wash store with washing fluid that is applied in a similar manner and that further enhances the results of the test. The devices and methods may be used with a variety of lateral flow assays, including assays based on changes in electrical property, assays based on optical detection, and others.

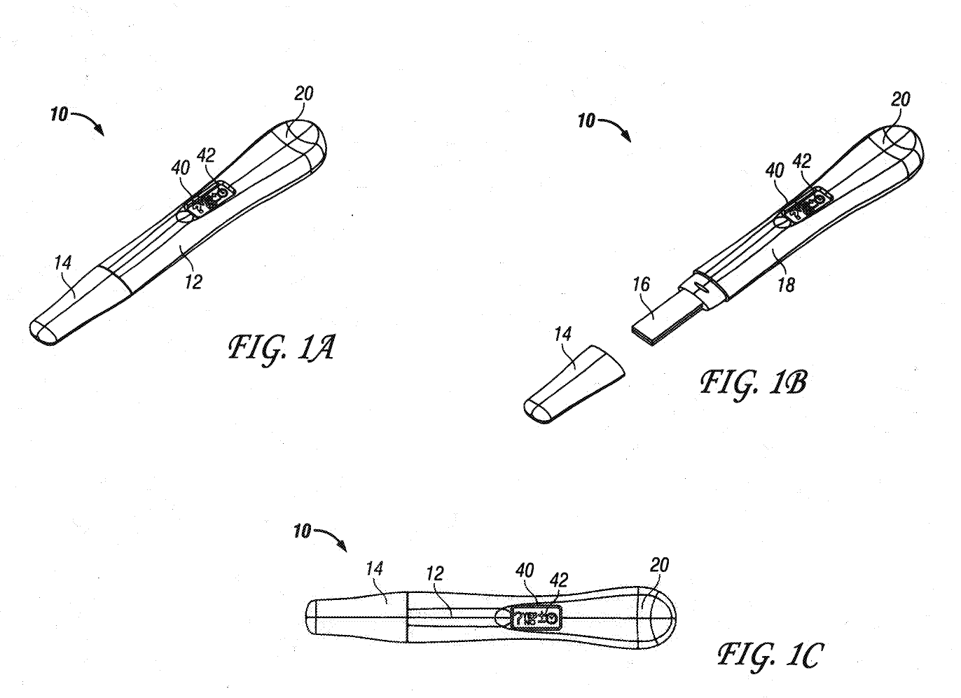

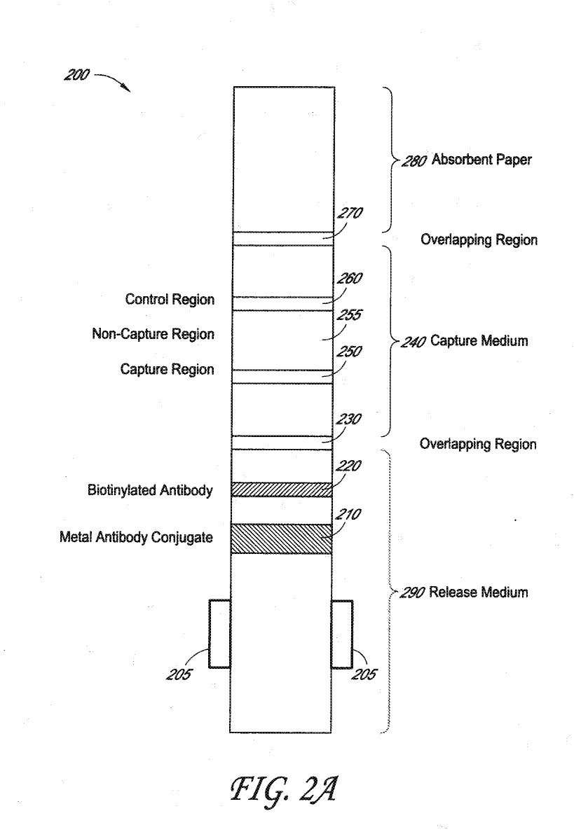

FIGS. 1A-C illustrate different views of an embodiment of an exemplary device 10. FIG. 1A illustrates a perspective view of the device 10 with a cap 14 intact, while FIG. 1B illustrates a perspective view of the device 10 with the cap 14 removed. FIG. 1C illustrates a top view of the device 10 with the cap 14 intact. The device 10 also comprises an outer, molded casing 12 which defines a hollow, elongate enclosure The casing 12 is configured to provide a recessed portion 20 shaped to permit users to place their thumb into the recessed portion and their forefinger on the bottom of the casing to securely hold the device 10. A central section on the top of the casing 12 defines a centrally located window 40 which permits a user to observe displayed test results. Inside the casing 12 is a lateral flow test strip and electronic components, details of which will be described further below. The casing 12 contains a sample receiving member 16 onto which a liquid sample can be applied to the test strip in the device 10. The sample receiving member 16 may therefore be or have a sample receiving region to which a fluid sample is applied. The removable cap 14 can be secured to one end of the casing 12 over the sample receiving member 16. A sample receiving member 16 is positioned so that part of the sample receiving member 16 is received in the enclosure defined by the casing 12 and part of the sample receiving member 16 extends from the end of the enclosure defined by the casing 12.

In some embodiments, changes in impedance are sensed electronically, as is discussed in further detail below, and the results are presented to a user on a display 42. The display 42 may render various icons or messages to a user, such as test results, device status, error messages, etc. The display 42 may be color or monochrome. In one embodiment, the display 42 is a liquid crystal display (LCD).

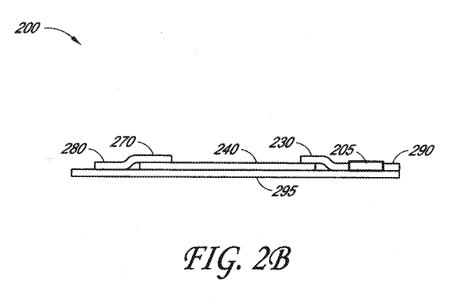

FIG. 2A is a top view of a diagram of an embodiment of a triphasic test strip suitable for use in an implementation of the invention, although it will be appreciated that a wide variety of test strip designs may be used. The fluid path along the test strip 200 will be discussed starting with the bottom of FIG. 2A as shown and moving up. This spatial orientation is merely a convenience for the sake of description.

At the bottom of the test strip 200, a fluid sample may be applied to the strip's release medium 290. In some embodiments, the fluid sample is applied to a sample receiving region, such as, for example, the sample receiving member 16 shown in FIG. 1B. The release medium 290 may also have a sample receiving region to which the fluid sample, and/or one or more enhancement solutions (described in further detail below), are applied. The test strip 200, including the release medium 290 and sample receiving member 16, may be formed from an absorbent material to aid in the uptake of the fluid sample. The fluid sample may flow across the strip and encounter a conjugate region 210. In the embodiment shown, the conjugate region 210 is a colloidal gold antibody conjugate region where the antibody binds to the analyte of interest (e.g. LH) present in the fluid sample. As the fluid sample passes through the conjugate region 210, analyte in the fluid sample will bind the gold-conjugated antibody in the liquid phase and carry the conjugate-analyte complex along the strip. While the embodiments shown are in the context of antibodies conjugated with a metal label, other materials may be used. In some embodiments, polymers are used as the label. For instance, PEG-alated systems or dendrimers may be used as a label. Further, the binding may involve ligand-receptor interactions other than antibody-antigen interactions. For example, artificial receptors may be used in the lateral flow assay, such as aptamers.

The fluid sample may then flow through a second antibody region 220. In the embodiment shown, the second antibody region 220 includes biotinylated antibody that specifically binds to a different epitope on the analyte of interest than the gold-conjugated antibody, forming a “sandwich” of analyte and two antibodies—one antibody with colloidal gold and the other with biotin. The sandwich may then be carried further along the test strip across a first overlapping region 230. The area from the start of the test strip 200 to the first overlapping region 230 may generally be referred to as the release medium 290.

After or at the overlapping region 230, the fluid on the test strip 200 encounters a capture medium 240, which may be nitrocellulose, or the like. As the fluid sample continues along the test strip 200 in the capture medium 240, the sample may next encounter a capture region 250, which may be a test line, containing a capture agent. The capture region 250 may be a narrow or wide region spanning all or substantially all of the width of the strip 200. In some embodiments, the capture agent in the capture region 250 is avidin. The avidin is for binding the biotin on the second antibody to trap the sandwich (with the gold) at the capture region 250. The capture region 250 may become darker as more of the sandwich complexes are accumulated.

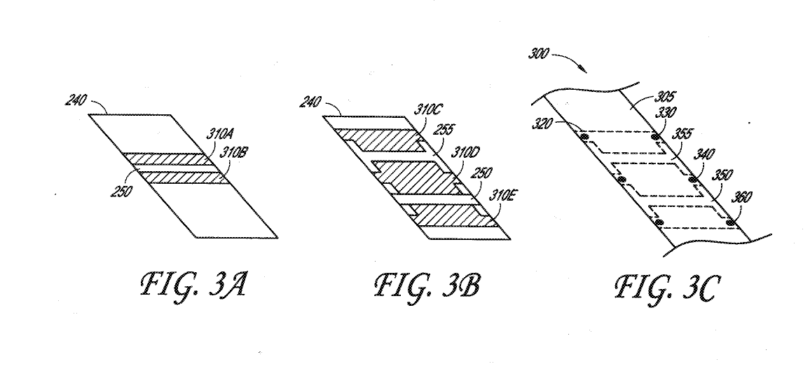

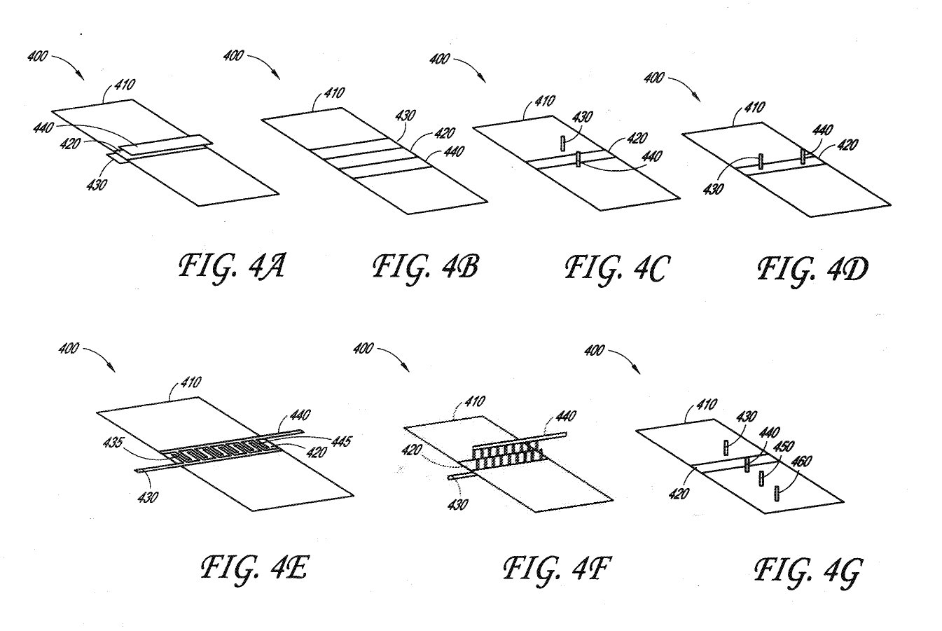

In an example implementation where the conjugate comprises colloidal metal such as, for example, gold or silver, an electrical system including electrodes and a processor (not shown in FIG. 2A) may measure effects, such as hydroscopic effects, of the colloidal gold specifically bound at the capture region 250 of the test strip 200. The processor may perform a transformative algorithm on the electrical signals detected by the electrodes. As is discussed in further detail herein for example with respect to FIGS. 3A-3C and 4A-4G, the electrodes may be in a number of different configurations, including on the strip 300 itself or on a circuit board that is assembled so the electrodes make contact with a conductive area around the capture region 250, and the processor may use electrical signals detected by the electrodes to measure an impedance or other electrical property associated with the capture region 250.

After the capture region 250, the test strip 200 includes a non-capture region 255. The non-capture region 255 is a region of bare membrane material of the strip 200 across which an electrical property such as impedance is measured. The non-capture region 255 is shown unmarked. However, the non-capture region 255 may be indicated by one or more lines, similar to the embodiment of the capture region 250 as shown. In some embodiments, the non-capture region 255 is bare membrane. The bare membrane region need not be a line shape, but may also be a strip or any shape. The region 255 may further be located anywhere along the length of the strip 200, bare membrane or otherwise, where there is no capture agent or striping reagent. The non-capture region 255 may thus be below the capture region 250 on the strip 200 (as oriented in FIG. 2A), such that the fluid sample encounters the non-capture region 255 before the capture region 250.

Electrodes (not shown in FIG. 2A) may be positioned in proximity to the non-capture region 255. The electrodes detect electrical signals to be used by the processor to measure an electrical property such as impedance across the non-capture region 255. As is discussed in further detail herein, for example with respect to FIGS. 3A-3C and 4A-4G, the electrodes may be in a number of different configurations, including on the strip 300 itself and/or on a circuit board that is assembled so the electrodes make contact with a conductive area around the capture region 250, and impedance or other measurements from electrodes in the capture region 250, and optionally the non-capture region 255, may be used to detect analyte presence and/or quantify analyte amount in the fluid sample.

After the capture region 250 and non-capture region 255, the test strip 200 may include a control region 260. The control region 260 may also generally be referred to as a reference region or reference line. When present, the control region 260 includes antibodies or other proteins that specifically bind the gold-conjugated antibody to provide a measurement of gold-bound antibody in the fluid that is not specifically bound to the analyte. Impedance measurements from the capture region 250 and/or control region 260 may be used separately to define successful testing. Strips without a control region 260 may be advantageous because it eliminates the need for the antibodies at this region as well as reduces complexity of the electrical system, thus reducing cost of the strip.

In embodiments with a control region 260, electrodes (not shown in FIG. 2A) may be positioned in proximity to the control region 260. The electrodes detect electrical signals to be used by the processor to measure an electrical property such as impedance across the control region 260. As is discussed in further detail herein, for example with respect to FIGS. 3A-3C and 4A-4G, the electrodes may be in a number of different configurations, including on the strip 300 itself and/or on a circuit board that is assembled so the electrodes make contact with a conductive area in and/or around the capture region 250.

In some embodiments, measurements from multiple non-capture, capture and/or control regions 255, 250, 260 on the same strip 200 may be taken. Therefore, there may be multiple sets, such as pairs, of electrodes in each or in some of the regions 255, 250, 260. Such configurations may reduce the chances of false negatives by sampling over a larger area. This may also mitigate the effects of localized abnormalities, defects or other causes of changes to the strip properties that may, for instance, be introduced during the preparing, making and/or life of the strip.

The capture medium 240 may interface with a second overlapping region 270. The second overlapping region 270 may serve as a border between the capture medium 240 and an absorbent portion 280 of the test strip. The absorbent portion 280 of the test strip 200 facilitates the uptake of the fluid sample as it arrives at the end of the test strip 200.

The various overlapping regions 230, 270 may be in a number of different configurations. For instance, the release medium 290 may be on top of the capture medium 240 at the overlapping region 230, or vice versa. Similarly, the capture medium 240 may be on top of the absorbent portion 280 at the overlapping region 270, or vice versa. Still other configurations of the overlapping regions 230, 270 within the ordinary skill of the art are contemplated and are within the scope of the present disclosure.

In some embodiments, the device 10 includes one or more stores 205. The stores 205 may be pots, blisters, reagent stores, or other compartments that can hold a fluid. In some embodiments, the one or more stores 205 contain enhancement solution(s). The solution(s) from the stores 205 allow for enhancement of a signal indicative of the results of the assay. This signal may be a visual signal, in which case the visual signal is enhanced. For example, the capture region 250 may become darker once the solution from the store 205 has flowed to the capture region 250. In another example, the measurement of an electrical property, such as impedance, capacitance, or resistance, may be enhanced once the solution from the store 205 has flowed to the region that is being measured. It is noted that these are merely illustrative examples, and other implementations and application involving using the one or more stores 205 are within the scope of the present disclosure.

As shown in FIG. 2A, the device 10 may include two stores 205. In some embodiments, one or more than two stores 205 are included. As shown, each store 205 is located on a side of the release medium 290. However, the stores 205 may be located in other areas in, on, above/below, or otherwise proximal to the strip 200. In some embodiments, the stores 205 may be located over the strip 200. In some embodiments, the stores 205 may be located partially over and partially to the side of the strip 200. As shown, the stores 205 may be located upstream of the capture region 250. By “upstream” of the capture region 250 it is meant a location that receives the flowing fluid sample before the capture region 250. In some embodiments, the stores 205 may be located upstream of the capture region 250, of the non-capture region, and/or of the control region 260. In some embodiments, the stores 205 may be located at or proximal to the sample receiving region of the strip 200. In some embodiments, the stores 205 may be located closer to the measurement region. In some embodiments, the stores 205 may be located over, in, on, adjacent or otherwise near the capture region 250.

The one or more stores 205 may contain one or more enhancement solutions. In some embodiments, a first store contains a first enhancement solution and a second store contains a second enhancement solution. In some embodiments, the enhancement solutions may comprise a silver salt solution and/or a hydroquinone initiator. In some embodiments, the enhancement solutions may contain chloroauric acid (HAuCl₄) and/or Hydroxylamine hydrochloride (NH₂OH.HCl). For example, the first store may include a first enhancement solution that comprises the silver salt solution or the like, and the second store may contain the second enhancement solution that comprises the hydroquinone initiator or the like. As another example, the first store may include a first enhancement solution that comprises chloroauric acid (HAuCl₄) or the like, and the second store may contain the second enhancement solution that comprises Hydroxylamine hydrochloride (NH₂OH.HCl) or the like.

The one or more stores 205 may contain one or more washing fluids that may be released from the one or more stores 205. In some embodiments, the washing fluids maybe released from the one or more stores 205 in the same manner as the solution enhancement(s) released form the stores. It is therefore understood that any features and/or functionalities of the stores with respect to enhancement solutions apply equally to stores having washing fluids.

The stores may further contain one or more releases. The release or releases may be actuated in order to cause or allow the enhancement solution inside a store to exit the store and flow onto or toward the strip. The solution from the stores may flow via capillary flow to the measurement region.

In some embodiments, the release may be a mechanical pusher configured to push the first enhancement solution from the at least one store and toward the strip. For example, the mechanical pusher may be a plunger or other structural member that is slid or otherwise moved to push or otherwise encourage the enhancement solution to exit the store. This is merely an example, and many other structures and mechanisms may be employed as a mechanical pusher.

In some embodiments, the release may be a puncture member. The puncture member may be configured to puncture the store and thereby cause the enhancement solution to exit the store and flow onto or toward the strip. For example, the puncture member may be a needle, poker, rod or other structural member that is slid or otherwise moved to puncture or otherwise break a portion or all of the store, Puncturing the store may cause or otherwise encourage the enhancement solution to exit the store and flow onto or toward the strip. This is merely an example, and many other structures and mechanisms may be employed as a puncture member.

In some embodiments, the release may comprise at least one dissolvable port. The port may be coupled to the store. In some embodiments, the strip is further configured such that, when the fluid sample is received by the sample receiving region, the fluid sample flows to the port. In some embodiments, the port may be configured to dissolve after the fluid sample flows to the port. For example, contact between the fluid sample and the port may initiate a chemical reaction that causes the port to dissolve. In some embodiments, various materials of varying shapes and sizes may be used for the port so that the dissolving rate of the port is controlled. In some embodiments, dissolving the port may allow the enhancement solution contained by the port to be provided to the strip. In some embodiments, the enhancement solution flows toward or onto the strip after the port has been partially or completely dissolved. For example, the store may have a port facing the strip that dissolves when the fluid sample flows to the port, thereby allowing the enhancement solution contained by the store to flow toward the strip. This is merely an example, and many other configurations may be employed as a dissolvable port.

In some embodiments, the release may be actuated by a user of the device. For instance, the mechanical pusher, the puncture member, or any other release may be actuated by a user of the device. For example, the casing 12 or portions thereof may have a release coupled with it such that movement of the casing 12 or portions thereof will actuate the release. In some embodiments, a user alters the casing 12 or portion thereof to a position other than its initial position after applying a fluid sample to the receiving region in order to actuate the release. For example, a user may slide, depress, pull, push, shake, or otherwise influence the casing 12 or portions thereof to actuate the release after the fluid sample has been applied to the receiving region. In some embodiments, a user depresses a button to puncture a store. In some embodiments, a user slides a mechanical pusher to break the store.

FIG. 2B is a side view of an embodiment of a triphasic test strip 200. The strip 200 has a mylar backing 295. The release medium 290 is coextensive with an end of the mylar backing 295. The release medium 290 overlaps with a portion of the capture medium 240 at overlapping region 230. An opposite portion of the capture medium 240 overlaps with the absorbent paper 280 at overlapping region 270. While the capture medium 240 is shown underneath the release medium 290 and absorbent paper 280, it may also be on top, or combinations thereof.

Further shown in FIG. 2B is an embodiment of the store 205. As shown, the store is coupled with the strip 200. In some embodiments, the store 205 is coupled to other parts of the device 10. In some embodiments, the store 205 is coupled with the casing 12. For example, the store 205 may be coupled with the inside of the casing 12. In some embodiments, the store 205 is mechanically attached to the strip 200 or casing 12. In some embodiments, the store 205 may be adhered to the strip 200 or casing 12. In some embodiments, the store 205 is screwed to the casing 12. In some embodiments, the store 205 is coupled with the casing 12, or parts thereon, such that when the casing 12 is assembled with the strip 200, the store 205 is located on, next to or otherwise adjacent the strip 200. As shown, the store 205 is coupled with the side of the strip 200 at the release medium 290. In some embodiments, the store 205 is coupled with the side of the fluid sample receiving region. These are just some examples and embodiments of the how the store 205 may be coupled with various parts of the device 10, and other configurations and implementations of the store 205 with the device 10 are within the scope of the present disclosure

FIGS. 3A-3B show perspective views of various embodiments of conductive areas about measurement regions on a test strip that may be used in an assay device that contains the stores with enhancement solution. In some embodiments, the assay is an impedance-based assay in which the impedance or other electrical properties are measured for one or more measurement regions of the strip. In some embodiments, the assay is an impedance-based assay that contains the conductive areas.

Referring to FIG. 3A, the capture medium 240 is shown having the capture region 250. The capture region 250 may span the entire width of the capture medium 240. In some embodiments, the capture region 250 may span less than the entire width of the capture medium 240. On both sides of the capture region 250 may be conductive areas 310A, 310B. The conductive areas 310A, 310B may be areas of the strip 200 adjacent to or otherwise near a measurement region, such as the capture region 250 or the like, with enhanced conductivity. In some embodiments, the conductive areas 310A, 310B may be adjacent to or otherwise near the non-capture region 255 and/or the control region 260.

The conductive areas 310A, 310B may include a variety of conductive materials. In some embodiments, the conductive areas 310A, 310B are screen printed materials. For instance, the conductive area may be a screen printed electrode, mesh, film or membrane. In some embodiments, the conductive areas 310A, 310B are a screen printed electrode based on carbon, gold, platinum, silver, carbon nanotubes ink, or combinations thereof. In some embodiments, the conductive areas 310A, 310B include carbon black, or variations thereof.

In related aspects, the conductive areas 310A, 310B may be in contact with electrodes from other parts of the device 10. For instance, when the device 10 is assembled, electrodes from a circuit board (e.g. a printed circuit board, or the like) may be in contact with the conductive areas 310A, 310B to measure impedance across the capture region 250.

FIG. 3B shows an exemplary embodiment of conductive areas about two measurement regions on a test strip. As shown, the capture medium 240 has the capture region 250, the non-capture region 255 and three conductive areas 310C, 310D, 310E. The conductive areas 310C, 310D, 310E may be in between and/or on either side of the two measurement regions. As shown, a first conductive area 310C is adjacent to the non-capture region 255, a second conductive area 310D is in between the capture region 250 and the non-capture region 255, and a third conductive area 310E is adjacent to the capture region 250.

The various conductive areas 310A-E may have various shapes and sizes. As shown in FIG. 3A, the conductive areas 310A, 310B may be rectangular and span the entire width of the capture medium 240. As shown in FIG. 3B, the conductive areas 310C, 310D, 310E may span the entire width of the capture medium 240 but have notches, or the like. For example, the notches may be adjacent to the measurement regions, i.e. the capture region 250 and the non-capture region 255. Having notched regions of the various conductive areas may facilitate reducing noise and similar effects due to non-uniformities of the strip near the edges. Other shapes of the various conductive areas may further be implemented (e.g. depending on the particular application) and are within the scope of the present disclosure.

FIG. 3C shows a partial perspective view of an embodiment of a circuit board or substrate with electrodes that may be used with the test strip of FIG. 3B. Other configurations of boards or substrates may be used with test strips, such as that shown in FIG. 3A. As shown in FIG. 3C, a portion of a circuit board 305 is visible having various electrodes 330, 340, 360, and others. The electrodes 330, 340, 360 may be a variety of types having different sizes, shapes, configurations, orientations, etc. For example, the electrodes 330, 340, 360 may be pins, pads, membranes, etc. The electrodes 330, 340, 360 may be in a pattern. In related aspects, the pattern may be complementary to the layout of the capture region 250, non-capture region 255, and/or the various conductive areas, such as those on the capture medium 240 as shown in FIG. 3B. Dotted lines 320 in FIG. 3C outline the various regions that match various conductive areas of the capture medium 240 in FIG. 3B. As shown in FIG. 3C, the dotted lines 320 define a non-capture outline 355 (which matches the shape of non-capture region 255) as well as a capture outline 350 (which matches the shape of the capture region 250). The dotted lines may further define regions that match up with the conductive areas 310C, 310D, and/or 310E. The pattern of electrodes may be based on these various areas defined by the dotted lines 320. A first electrode 330 and second electrode 340 are located adjacent to the non-capture outline 355 in locations that would match up with corresponding first and second conductive areas 310C and 310D on the strip 200. Similarly, a third electrode 360 is located adjacent to the capture outline 350 in locations that would match up with corresponding third conductive area 310E on the strip 200.

When the board 305 of FIG. 3C contacts the capture medium 240 of FIG. 3B, the electrodes 330, 340, 360 may each be located, oriented, positioned or otherwise configured to contact a respective conductive area 310C, 310D, 310E on the capture medium 240. Thus, the impedance or other electrical properties (from which the impedance may be derived) may be measured across the various measurement regions using conductive pathways that are formed from the conductive areas 310C, 310D, 310E to one of the respective contacting electrodes 330, 340, 360. Because the conductive areas 310C, 310D, 310E abut the measurement regions, the signal is indicative of the impedance or other electrical property across the measurement region. In some embodiments, the board 305 of FIG. 3C is turned over and placed on top of the capture medium 240 as shown in FIG. 3B. Further, other electrodes are shown in FIG. 3C which may be used instead of or in addition to electrodes 330, 340, 360. In some embodiments, multiple measurements across the same measurement region are taken for enhanced accuracy and reliability of results.

FIGS. 3A-3C demonstrate embodiments for measuring various electrical properties across the measurement regions with electrodes that are brought to contact conductive areas on the strip. However, other embodiments include electrodes embedded in the test strip 300 that would then be connected to the circuit board 305. Regardless of whether the electrodes are part of the board 305 and brought into contact with the strip 300 when assembled, or whether the electrodes are embedded in the strip 300, the electrodes may have numerous layouts and patterns and be of varying shapes, sizes and orientations. Some of the possible layouts and designs for the electrodes are discussed with respect to FIGS. 4A-4G.

FIGS. 4A-4G show embodiments of layouts 400 of electrodes 430, 440 in the proximity of a measurement region 420 on a strip portion 410 that may be implemented in a test strip 200 in the assay device 10. In some embodiments, the strip portion 410 may be the capture medium 240 of FIGS. 2A, 2B, 3A, and/or 3B. The measurement region 420 may be the capture region 250, the non-capture region 255 and/or the control region 260. While FIGS. 4A-4G show embodiments of the electrode layouts on a strip portion 410, it is understood that the layouts shown and discussed below may also be embodied on the board 305 (see FIG. 3C). Thus, the electrode layouts discussed in the context of being embedded in, on or with the strip portion 410 may also be in embodiments where the electrodes are on a circuit board and are brought into contact with the conductive areas 310 (see FIGS. 3A-3B).

FIGS. 4A and 4B show layouts 400 of electrodes 430, 440 as bands or wires that span all or substantially all of the width of the strip portion 410 near the measurement region 420. The electrodes 430, 440 may be less wide, for example a wire, or wider, for example a band. As shown in FIG. 4A, the electrodes 430, 440 may be respectively below and above the measurement region 420. Electrode 440 is on top of the strip portion 410 over the measurement region 420, while electrode 430 is below the strip portion 410 under the measurement region 420. As shown in FIG. 4B, the electrodes 430, 440 may be on either side or edge of the measurement region 420. Electrode 440 is on one side of the measurement region 420, while electrode 430 is on the opposite side of the measurement region 420. The electrodes 430,440 need not be near either side of the measurement region 420, but rather the electrodes 430,440 may be away from one or both sides of the measurement region 420.

FIGS. 4C and 4D show layouts 400 of electrodes 430, 440 as pin electrodes near the measurement region 420. The electrodes 430, 440 may be configured on top, through, or partially through the strip portion 410. As shown in FIG. 4C, the electrodes 430, 440 may be on either side of the measurement region 420. Electrode 440 is on one side of the measurement region 420, while electrode 430 is on the opposite side of the measurement region 420. As shown in FIG. 4D, the electrodes 430, 440 may be configured along the measurement region 420. Electrode 440 is on one end of the measurement region 420, while electrode 430 is on the opposite end of the measurement region 420.

FIG. 4E shows a layout 400 of electrodes 430, 440 as interdigitated electrode arrays having electrode teeth 435, 445 that span all or substantially all of the width of the strip portion 410 near the measurement region 420. Interdigitated electrode arrays may be used to improve the signal to noise ratio of the electrical property being measured. The teeth 435, 445 of array electrodes 430, 440 may be micro- or macro-scale. The electrodes 430, 440 and/or electrode teeth 435, 445 may span less than the width of the strip portion 410. As shown, the electrodes 430, 440 may be respectively below and above the measurement region 420. Electrode 440 is on top of the strip portion 410 over the measurement region 420, while electrode 430 is below the strip portion 410 under the measurement region 420.

FIG. 4F shows a layout 400 of electrodes 430, 440 as comb electrodes that span all or substantially all of the width of the strip portion 410 through the measurement region 420. The electrodes 430, 440 may be arrays of pins mounted through the measurement region 420 from either side of the strip portion 410. The electrodes 430, 440 may span less than the width of the strip portion 410. As shown, the electrodes 430, 440 may be respectively below and above the measurement region 420. Electrode 440 is on top of the strip portion 410 over the measurement region 420, while electrode 430 is below the strip portion 410 under the measurement region 420.

FIG. 4G shows a layout 400 of electrodes 430, 440, 450, 460 as pin electrodes both near and away from the measurement region 420. The electrodes 430, 440, 450, 460 may be pins or bands. Polling different pairs of the electrodes 430, 440, 450, 460 allows for simpler manufacturing as the measurement region 420 does not require precise placement on the strip portion 410. It also allows for improved measurements through algorithms that analyze data from different pairs of the electrodes 430, 440, 450, 460. Some embodiments may have multiple measurement regions 420 each with one or more electrodes.

The electrodes associated with the strip portion 410 and strip 200 may be integrated with an electrical system including a processor. In some embodiments, the electrical system may be used to actuate one or more releases. In some embodiments, the electrical system may be used to produce and analyze electrical property measurements, such as impedance, of the various measurement regions 420.

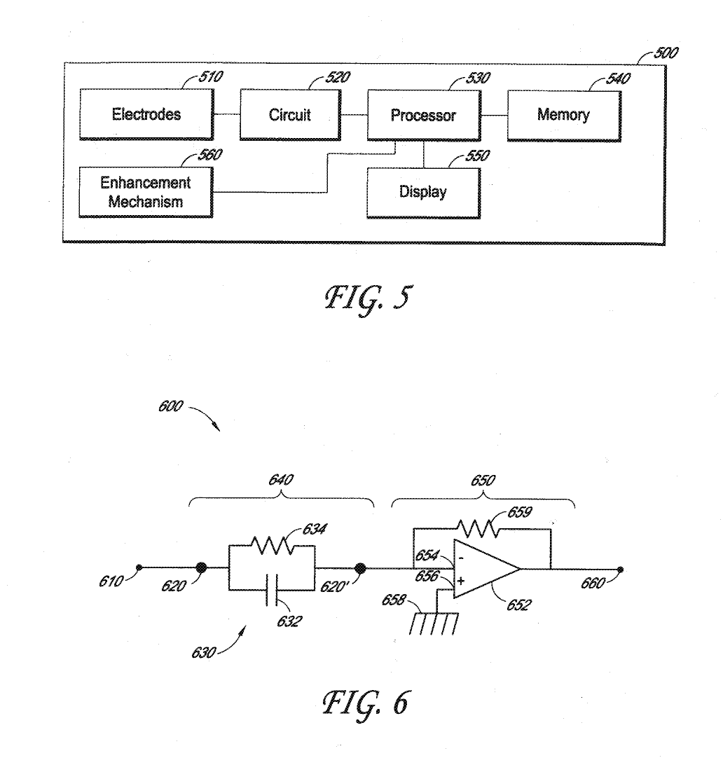

FIG. 5 shows a block diagram of an embodiment of an electrical system 500 that may be in the assay device 10 having the enhancement solution. In one example, the system 500 may include an enhancement mechanism 560. The enhancement mechanism 560 may include one or more stores 205. In some embodiments, the enhancement mechanism 560 may include one or more stores 205 with enhancement solution(s). In some embodiments, the enhancement mechanism 560 may include one or more stores 205 with one or more releases. In some embodiments, the enhancement mechanism 560 may include one or more stores 205 that contain enhancement solution(s) and that are coupled with one or more releases. In some embodiments, the enhancement mechanism 560 may include one or more releases that are actuated a set time after the sample receiving region receives the fluid sample. In some embodiments, the enhancement mechanism 560 includes one or more mechanical pushers configured to be actuated by an operatively coupled processor 530 and thereby push one or more enhancement solutions from one or more stores and toward the strip. In some embodiments, enhancement mechanism 560 includes one or more puncture members configured to be actuated by the operatively coupled processor 530 and thereby puncture the one or more stores to cause and/or allow one or more enhancement solutions from one or more stores to flow toward the strip.

The system 500 may include electrodes 510. The electrodes 510 may be conductive materials associated with the measurement regions 420 and connected to a circuit 520. The circuit 520 transmits electrical signals to a processor 530. The processor 530 may analyze the signals in order to measure one or more electrical properties, such as the impedance, of the regions 420. In some embodiments, the release is actuated when a measured electrical property of a measurement region reaches and/or surpasses a threshold value. In some embodiments, the release is actuated after the measured impedance of the capture region reaches a threshold impedance. The processor 530 is also connected to a memory 540. The memory 540 may contain a set of instructions for the processor 530 to carry out in order to measure the impedance. The memory 540 may also store other digital data, such as records of impedance measurements, data correlations, lookup tables, etc. The processor 530 is also connected to a display 550. The processor 530 may send signals to the display 550, which may be a digital display, in order to show information related to the various processes performed. For instance, the display may indicate a power status of the device 10, that a measurement is currently being taken, or the results of a measurement such as the presence and/or quantity of an analyte, or the non-presence of an analyte, in a fluid sample.

The circuit 520 and electrodes 510 of FIG. 5 may be implemented in a number of configurations in a device 10. FIG. 6 shows a circuit diagram of an embodiment of a circuit 600 including electrodes 620. As shown, the circuit 600 includes a strip region 640 that may be, for example, the measurement region 420 across the strip 410 of FIGS. 4A-4G. In FIG. 6, a voltage may be applied to the circuit 600 with a voltage source at node 610. The voltage may be applied over a range of frequencies. In some embodiments, the range is 1 KHz to 100 KHz. Current flows through the circuit 600 to the first electrode 620 in the strip region 640.

After first electrode 620, current flows through the strip region 640 and to a second electrode 620′. The strip region 640 is represented as an RC circuit 630 with a resistor 634 and capacitor 632 in parallel. The amount of current flowing to the second electrode 620 will vary depending on the measured electrical property, such as the impedance, of the strip region 640. The impedance corresponds to and is a measure of the opposition that the strip region 640 presents to the current flowing through it. If the impedance is high, less current will flow through the strip region 640, and vice versa. Thus other related electrical concepts and properties are applicable to and may be measured in the present disclosure, such as resistance and capacitance.

In some embodiments, the amount of colloidal metal in the strip region 640 influences the intensity of the measured electrical property. In some embodiments, one or more enhancement solutions may be added to the strip region 640 to enhance the intensity of the measured electrical property. In some embodiments, a silver salt material and an initiator such as hydroquinone solution are added to introduce more silver to the strip region 640 to enhance the intensity of the measured impedance of the strip region 640. In some embodiments, chloroauric acid (HAuCl₄) and Hydroxylamine hydrochloride (NH₂OH.HCl) are added to introduce more gold to the strip region 640 to enhance the intensity of the measured impedance of the strip region 640.

After flowing through the strip region 640, the current encounters the second electrode 620. The second electrode 620 is connected to a current to voltage converter 650, to which the current next flows. The convertor 650 includes an operational amplifier or op-amp 652 and a feedback resistor 659. The op-amp 652 has a non-inverting input 654 and inverting input 656 connected to a ground 658. The current flows to the converter 650 and is output as a voltage at node 660. The output voltage at node 660 compared to the input voltage at node 610 provides a measurement of the electrical property, such as the impedance, associated with the strip region 640. The electrical property, such as the impedance, may be indicative of the presence and quantity of analyte in the region 640. In some embodiments, the output voltage may be increased due to the presence of one or more enhancement solutions and/or washing fluids.

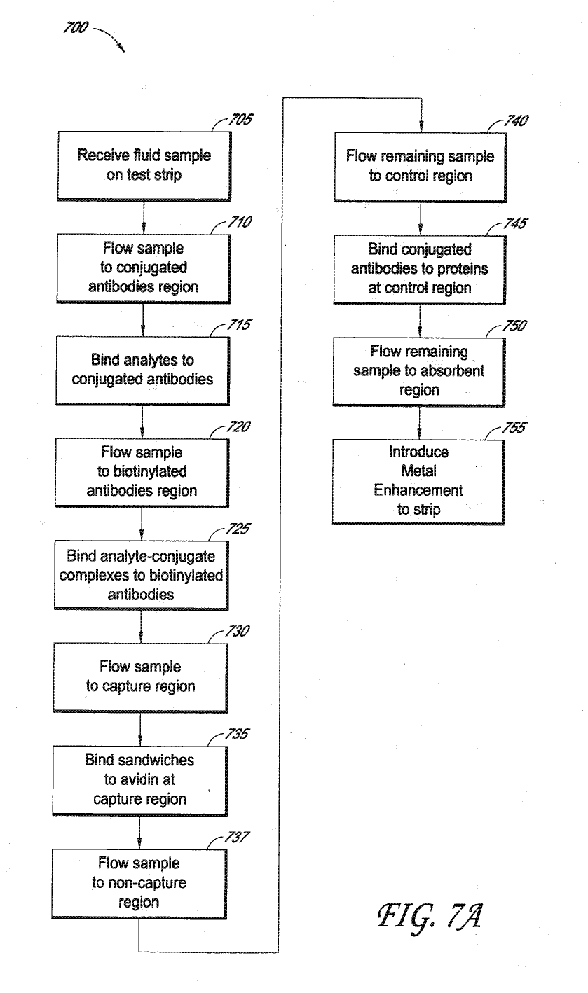

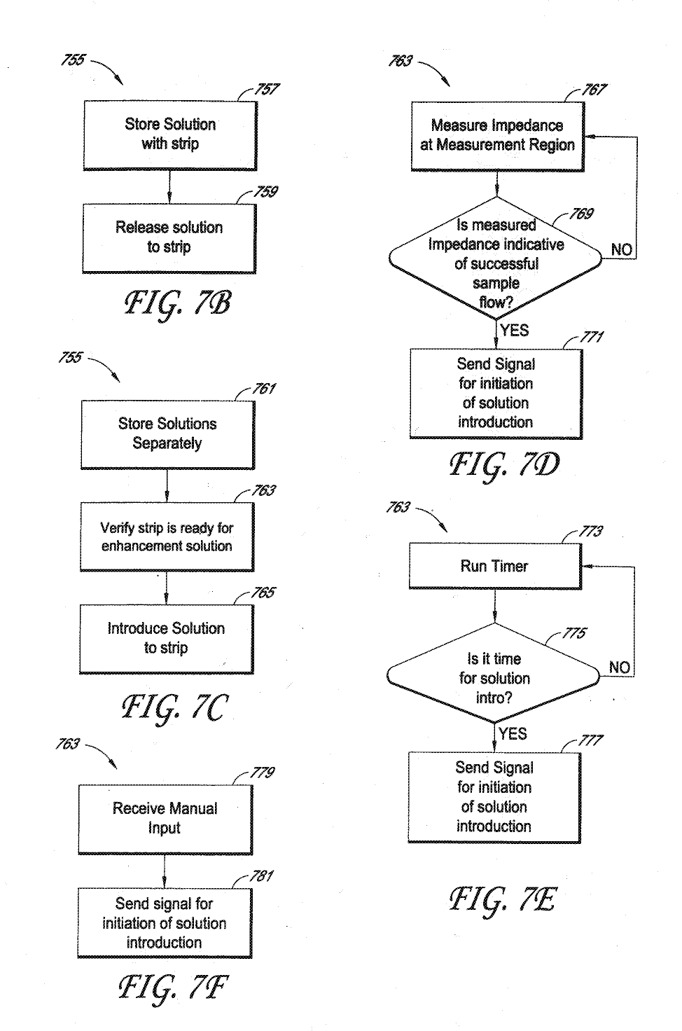

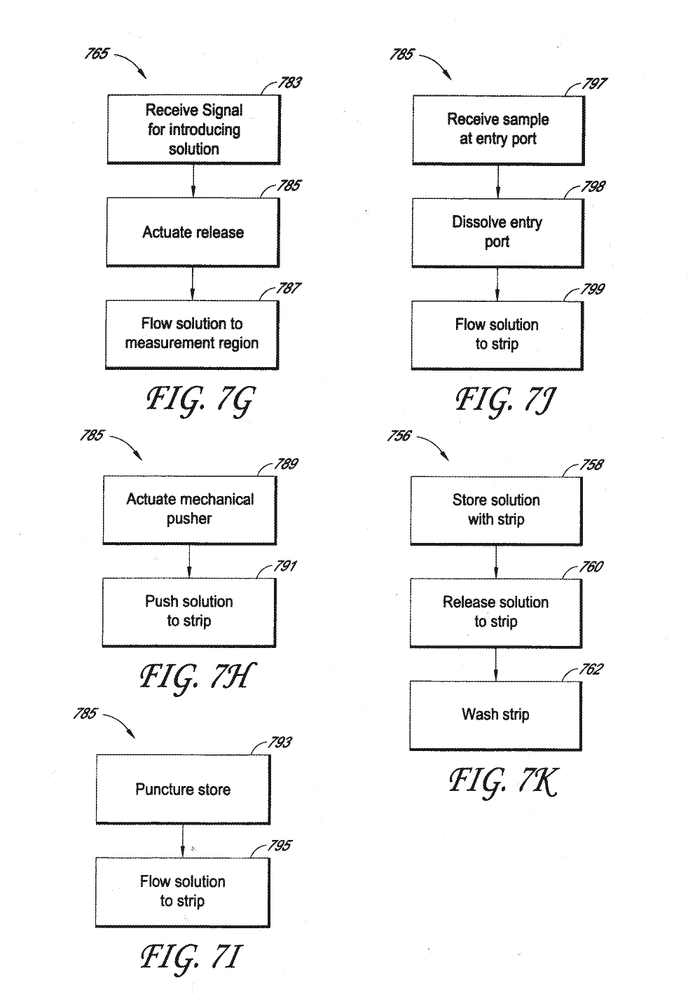

FIG. 7A shows a flow chart of an embodiment of a method 700 of providing enhancement solution to a strip after applying a fluid sample to a receiving member or portion on the strip. The process may begin with step 705 where a fluid sample is received on a test strip. The fluid sample may be received at the sample receiving member 16 or release medium 290. Next, in step 710, the fluid sample flows to a conjugate antibodies region, which may be the conjugate region 210. At the conjugate antibodies region, analytes in the fluid sample, if present, are bound to the conjugated antibodies in the next step 715. The fluid sample then flows to a biotinylated antibodies region, which may be the second antibody region 220. At the biotinylated antibodies region 220, the analyte-conjugate complexes are bound to the biotinylated antibodies in step 725. Thus a “sandwich” of analyte and two antibodies, one with colloidal gold and the other with biotin, are formed and continue flowing along the strip. Next, in step 730, the fluid sample flows to a measurement region or test line, which may be the capture region 250. En route to the test line, the sample may encounter an interface between the release medium and the capture medium, such as the overlapping region 230. At the test line, the sandwiches in the fluid sample are bound to avidin or the like and captured at the test line. In some embodiments, the method 700 then moves to step 740 wherein the fluid sampled flows to a control line, such as the control region 260. Then, in step 745, conjugated antibodies are bound to proteins at the control line. From the control line, the remaining fluid sample in step 750 flows to the absorbent region. In some embodiments, block 740 may be skipped where there is no control line.

The process 700 may further include step 755 where solution enhancement is introduced to the strip, such as the strip 200 (e.g., upstream of the capture region 250). As shown, step 755 may include introducing metal enhancement, such as gold, silver, or other metals. For example, a silver salt solution, a hydroquinone initiator, chloroauric acid (HAuCl₄), and/or Hydroxylamine hydrochloride (NH2OH.HCl) may be introduced in step 755. In some embodiments, step 755 may include introducing a washing fluid. Step 755 is discussed in further detail herein, for example with reference to FIGS. 7B-7C.

FIGS. 7B-7C show flow charts of different embodiments of method 755 for introducing enhancement solution to a strip. The method 755 may be incorporated into the method 700 shown in FIG. 7A, for example in step 755. FIGS. 7B-7K show further operations or aspects of the method of FIG. 7A that are optional and are not required to perform the method 700. If the method 700 includes at least one block of FIGS. 7B-7K, then the method 700 may terminate after the at least one block, without necessarily having to include any subsequent downstream block(s) that may be illustrated. The store or stores used in any or all of these methods may be the store 205 and the strip may be the strip 200.

Referring to FIG. 7B, method 755 may include step 757 where solution is stored with the strip. In some embodiments, one or more enhancement solutions are stored with a strip. The solutions may be stored in any of the manners discussed herein, for example in the store 205. Method 755 may further include step 759 where the enhancement solution is released to the strip. In some embodiments, one or more enhancement solutions are released from the stores and caused or otherwise allowed to flow to the strip. The solutions may be released in any of the manners discussed herein, for example by actuating a release using the system 500 shown in FIG. 5.

FIG. 7C is a flow chart of another embodiment of method 755 for introducing enhancement solution to a strip. As shown in FIG. 7C, the method 755 may include step 761 where enhancement solutions are stored separately. In some embodiments, a first solution is put into a first store and another solution is put into a second store. In some embodiments, the solutions are stored in the stores 205. In some embodiments, the first store and second store are separate so that the two solutions are kept separate and do not mix while inside their respective stores. Method 755 may further include step 763 where it is verified that the strip is ready for enhancement solution to be introduced. This may be verified in any of the ways discussed herein, such as by measuring electrical properties of measurement regions on the strip, waiting a set time after fluid sample application, by manual input after visual verification, and others. Further details of step 763 are discussed herein, for example with respect to FIGS. 7D-F. Method 755 may further include step 765 where enhancement solution is introduced to the strip. The solutions may be released in any of the manners discussed herein, for example by actuating a release using the system 500 shown in FIG. 5.

FIGS. 7D-7F are flow charts of different embodiments of a method 763 for verifying that a strip is ready for introducing enhancement solution. Method 763 may be used in step 763 of method 755 shown in FIG. 7C.

Referring to FIG. 7D, a method 763 is shown for verifying that a strip is ready for introducing enhancement solution using measurements of various electrical properties of the strip. In some embodiments, method 763 may be done using the system 500 shown in FIG. 5 and/or the circuit shown in FIG. 6. The method 763 may include step 767 where the impedance is measured at a measurement region of the strip 200. In some embodiments, other electrical properties may be measured, such as capacitance or resistance. In some embodiments, the electrical property is measured at the capture region 250, the non-capture region 255, the control region 260, and/or other regions of the strip 200. In some embodiments, the properties may be measured in step 763 using the processor 530 and electrodes 620 with the strip 200.

The method 763 may further include decision step 769 where it is determined whether the measured electrical property is indicative of successful fluid sample flow. As shown, step 769 may involve determining whether the measured impedance is indicative of successful fluid sample flow. In some embodiments, the measured electrical property is indicative of successful fluid sample flow when the property reaches a threshold value. For example, step 769 may include measuring the impedance of a measurement region of the strip to determine whether the impedance has reached a threshold impedance. In some embodiments, the fluid sample may alter the electrical property being measured for a measurement region. Therefore, measuring the electrical property may be indicative that the fluid sample has flowed to the measurement region. In some embodiments, step 769 may be performed with the system 500 shown in FIG. 5. For example, step 769 may be performed with a module in memory 540 that provides instructions that configure the processor 530 to compare the measured property with a threshold property stored in memory 540. If it is determined in decision step 769 that the measured electrical property is not indicative of successful fluid sample flow, then the method 763 may return to step 767. If it is determined in decision step 769 that the measured electrical property is indicative of successful fluid sample flow, then the method 763 may proceed to step 771.

The method 763 may further include step 771 where a signal is sent for initiating introduction of the solution. In some embodiments, step 771 may be performed with the system 500 shown in FIG. 5. For example, step 771 may be performed with a module in memory 540 that provides instructions that configure the processor 530 to send a signal to the enhancement mechanism 560 to actuate the release.

Referring to FIG. 7E, a method 763 is shown for verifying that a strip is ready for introducing enhancement solution using a set time. In some embodiments, method 763 includes step 773 where a timer is run. The timer may be a digital timer that counts the time that has passed. In some embodiments, the timer may be run after the fluid sample has been applied to the receiving region. For example, the system 500 may be used to detect a change in an electrical property, such as impedance, of a measurement region, such as the capture region, where the timer is run after a change in the electrical property is detected. In some embodiments, the impedance is measured as discussed above with respect to step 767 of method 763 shown in FIG. 7D. In some embodiments, the system 500 of FIG. 5 is used such that the electrical properties are measured at the receiving region, where the timer is run after a change in the electrical property at the receiving region is detected. In some embodiments, the timer may be run manually by a user. For example, a user may run a timer after applying a fluid sample to the receiving region.

The method 763 may further include decision step 775 where it is determined whether it is time to introduce one or more enhancement solutions. In some embodiments, it is determined whether it is time to introduce one or more enhancement solutions by comparing the timer that is run in step 773 with a set time. For example, when the timer run in step 773 reaches the set time, then it may be time to introduce the enhancement solution. In some embodiments, the system 500 and/or circuit 600 are used in step 773. For example, the memory 540 may have a module that provides instructions that configure the processor 530 to compare a running digital timer to a set time that is stored in the memory 540. If it is determined in decision step 775 that it is not time to introduce one or more enhancement solutions, then the method 763 may return to step 773. If it is determined in decision step 775 that it is time to introduce one or more enhancement solutions, then the method 763 may proceed to step 777.

The method 763 may further include step 777 where a signal is sent for initiation of solution introduction. Step 777 may be similar to step 771 as shown in and described with respect to FIG. 7D.

Referring to FIG. 7F, a method 763 is shown for verifying that a strip is ready for introducing enhancement solution using manual input. The method 763 may include step 779 where manual input is received. In some embodiments, step 779 may include physical input from a user of the device. For example, a user may physically actuate a release, such as a mechanical pusher or puncture member. In some embodiments, step 779 may include a user providing a digital or electrical signal. For example, a user may press a button on the device. In some embodiments, the system 500 shown in FIG. 5 may be used, such that a user may press a button on the device that initiates a module in memory 540 to provide instructions that configure the processor 530 to communicate with the enhancement mechanism 560. The method 763 may further include decision step 781 where a signal is sent for initiation of solution introduction. Step 781 may be similar to step 771 as shown in and described with respect to FIG. 7D.

Referring to FIG. 7G, a method 765 is shown for introducing a solution to a strip. In some embodiments, the method 765 may be used in step 765 of method 755 shown in FIG. 7C. The method 765 may include step 783 where a signal is received for introducing the solution. In some embodiments, step 783 includes receiving the signals that were sent in step 771 of FIG. 7D, in step 777 of FIG. 7E, or in step 781 of FIG. 7F. Referring to FIG. 7G, the method 765 may further include step 785 where one or more releases are actuated. The releases may be actuated in any number of ways, such as those described in further detail herein, for example with respect to FIGS. 7H-J. Referring to FIG. 7G, the method 765 may further include step 787 where the solution flows to the measurement region. In some embodiments, step 787 includes flowing a solution from a store to the strip. For example, the store 205 may have a release actuated and the enhancement solution inside the store 205 may exit and flow to the strip 200.

FIGS. 7H-7J are flow charts of different embodiments of methods 785 for actuating a release. In some embodiments, the methods 785 may be used in step 785 of method 765 shown in FIG. 7G.

Referring to FIG. 7H, the method 785 may include step 789 where a mechanical pusher is actuated. In some embodiments, step 789 includes sliding, raising, lowering, rotating, or otherwise moving the mechanical pusher such it pushes enhancement solution from a store and toward a strip. In some embodiments, step 789 includes the mechanical pusher breaking or otherwise breaching the store to cause or allow the enhancement solution to flow toward the strip. The method 785 may further include step 791 where the enhancement solution is pushed to the strip. In some embodiments, step 791 includes the mechanical pusher pushing the enhancement solution from the store and onto the strip. In some embodiments, step 791 includes the solution flowing through a breached store and onto the strip. In some embodiments, step 789 and/or step 791 includes use of the system 500 shown in FIG. 5. For example, a module in memory 540 may provide instructions that configure the processor 530 to actuate a mechanical pusher that is part of the enhancement mechanism 560 to push the solution from the store and toward and/or on the strip. In some embodiments, steps 789 and/or step 791 include manual operations performed by a user. In some embodiments, step 789 includes a user manually actuating a mechanical pusher. For example, steps 789 and 791 may include a user shifting or otherwise moving the casing 12 or portion thereof from an initial position to a shifted position such that a mechanical pusher coupled with the casing 12 is also shifted to push solution from the store 205 and toward the strip 200.

Referring to FIG. 7I, another embodiment of method 785 is shown. As shown, the method 785 may include step 793 where a store is punctured. In some embodiments, step 793 includes sliding, raising, lowering, rotating, or otherwise moving a puncture member. In some embodiments, step 793 includes moving the puncture member such that it breaks, breaches, opens, tears, pierces, rips, moves, or otherwise punctures the store or a portion thereof. The method 785 may further include step 795 where the enhancement solution flows to or toward the strip. In some embodiments, step 795 includes causing or otherwise allowing the enhancement solution to flow from the store and toward or to the strip. In some embodiments, step 795 includes the solution flowing through a punctured store and onto the strip. In some embodiments, steps 793 and/or step 795 include use of the system 500 shown in FIG. 5. For example, a module in memory 540 may provide instructions that configure the processor 530 to actuate a puncture member that is part of the enhancement mechanism 560 to puncture the store and allow the solution to flow toward the strip. In some embodiments, steps 793 and/or step 795 include manual operations performed by a user. For example, step 793 may include a user shifting or otherwise moving the casing 12 or portion thereof from an initial position to a shifted position such that a puncture member coupled with the casing 12 punctures the store 205 to allow solution to flow toward the strip 200.

Referring to FIG. 7J, another embodiment of method 785 is shown. As shown, the method 785 may include step 797 where a fluid sample is received at a port. In some embodiments, the port is coupled with a store. In some embodiments, the port is an entry port of a store through which enhancement solution may enter and/or exit. In some embodiments, step 797 includes a fluid sample applied at the receiving region of a device 10 flowing laterally to the port. In some embodiments, the port is a dissolvable port.

The method 785 may further include step 798 where the port is dissolved. In some embodiments, an entry port is dissolved. In some embodiments, the port is configured with a particular material or materials, shape, size, extent, orientation, position, and other parameters related to its configuration. In some embodiments, the port is configured such that the rate at which it dissolves is controlled in step 798. In some embodiments, step 798 includes a port dissolving over a pre-determined length of time. In some embodiments, step 798 includes a port dissolving such that the fluid sample has time to flow laterally to a measurement region. In some embodiments, step 798 includes a port dissolving such that the fluid sample has time to flow laterally to a capture region, non-capture region, and/or control region. For example, the store 205 of strip 200 may have a port configured to dissolve after a set time such that the fluid sample has enough time to flow to the capture region 250 within the set time. The one or more ports may dissolve after contact with the fluid sample. For example, contact between the fluid sample and the port may initiate a chemical reaction that causes the port to dissolve. In some embodiments, step 798 includes multiple ports dissolving. For example, step 798 may include a first port of a first store 205 dissolving as well as a second port of a second store 205 dissolving. In some embodiments with multiple ports, step 798 may include the ports dissolving simultaneously or nearly simultaneously. In some embodiments with multiple ports, step 798 may include the ports dissolving at different times. For example, a first port may be further upstream than a second port, where the fluid sample reaches the first port before the second port, causing the first port to dissolve before the second port. In another example, a second port may be configured differently than a first port, for instance with a different shape, size, material, etc., such that the two ports dissolve at different times.

The method 785 may further include step 799 where the solution flows to or toward the strip 200. In some embodiments, step 799 includes causing or otherwise allowing the enhancement solution to flow from the store and toward or to the strip. In some embodiments, step 799 includes the solution flowing through one or more dissolved ports and toward or onto the strip.

FIG. 7K is a flow chart of an embodiment of a method 756 for introducing a washing fluid to the strip. In some embodiments, method 756 may be performed to improve the quality of the results of a test performed with the assay device 10. In some embodiments, method 756 may be performed after an enhancement solution has been introduced to the strip. In some embodiments, method 756 may be performed after the method 700 of FIG. 7A has been performed. In some embodiments, method 756 may be performed after any of one or more of the methods discussed herein for introducing an enhancement solution to the strip have been performed, including, but not limited to, after any embodiments of methods 755, 763 and/or 785.

Referring to FIG. 7K, the method 755 may include step 758 where a solution is stored with the strip and step 760 where the solution is released to the strip. In some embodiments, the solution may be one or more washing fluids. In some embodiments, steps 758 and 760 may be similar to steps 757 and 759 of FIG. 7B where the solution is now a washing fluid. In some embodiments, one or more washing fluids are stored with the strip. The washing fluid may be stored in any of the manners discussed herein with respect to storing an enhancement solution, for example in the store 205.

The method 755 may further include step 760 where the enhancement solution is released to the strip. The washing fluid may be released to the strip in any of the manners discussed herein with respect to releasing an enhancement solution, for example step 759 of method 755 shown in FIG. 7B. In some embodiments, one or more enhancement solutions are released from the stores and caused or otherwise allowed to flow to the strip. The solutions may be released in any of the manners discussed herein, for example by actuating a release using the system 500 shown in FIG. 5.

The method 755 may further include step 762 where the strip is washed. In some embodiments, step 762 includes washing the strip with one or more washing fluids. In some embodiments, step 762 includes washing the strip with the washing fluid released in step 760. In some embodiments, step 762 includes flowing the washing fluid laterally along the strip. In some embodiments, step 762 includes flowing the washing fluid laterally to one or more measurement regions. For example, step 762 may include flowing the washing fluid laterally to the capture region 250, the non-capture region 255, and/or the non-capture region 260.

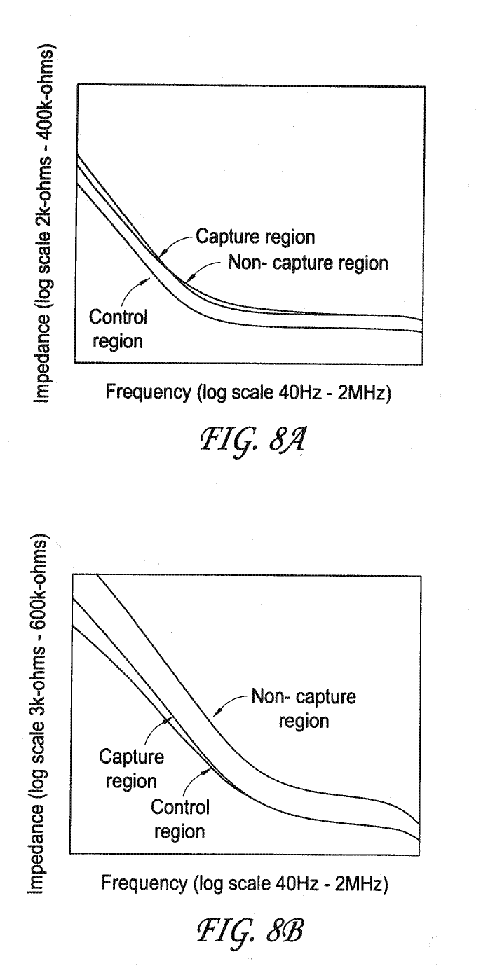

Referring to FIGS. 8A and 8B, the impedance values of the capture region, non-capture region and the control region at various sampling frequencies are shown. FIGS. 8A and 8B show impedance values on a log scale measured at various frequencies for a strip with no analyte and with analyte, respectively. Without any analyte, the capture region and non-capture region are very similar, as shown in FIG. 8A. However, as shown in FIG. 8B, with analyte the capture region has diverged from the non-capture region. With analyte, in some embodiments the capture region may be closer to the control region values than the non-capture values.

In some embodiments, the divergence of the capture and non-capture values may be compared to a threshold to determine the presence and quantity of an analyte. The divergence here means the difference in impedance for the capture and non-capture regions measured at a given frequency at a given time. A divergence that is greater than a threshold amount may indicate the presence of an analyte. It is noted that the divergence may be larger with the solution enhancement. It is also noted that the divergence may be larger with solution enhancement and washing fluid. For example, the difference between the capture and non-capture impedance values may be larger at a given frequency and given time in a lateral flow assay with solution enhancement as opposed to a lateral flow assay without solution enhancement.

In some embodiments, the measured impedances of the measurement regions may be larger with the solution enhancement. That is, not only may the divergence be larger for a positive test result, but the actual values of the impedances of the capture and non-capture regions may also be larger. This may make it easier to detect the impedances due to the larger values. It may also allow for a less complex and precise electrical system, such as system 500 in FIG. 5, to measure the electrical properties of a strip, leading to savings in cost of manufacturing and/or savings in cost to consumers.

In some embodiments, the enhancement solution and/or washing fluid may make it easier to visually verify the results of a test. For instance, the enhancement solution may make a measurement region more visible. For example, enhancement solution may be provided to a capture region and a control region, and for a positive and successful test the two regions may be darker as a result of the solution and thus easier to see. These are just some examples of types of lateral flow assays that may benefit from the provision of enhancement solution, and other types not explicitly mentioned herein may also benefit from other types of enhancement with the enhancement solution.

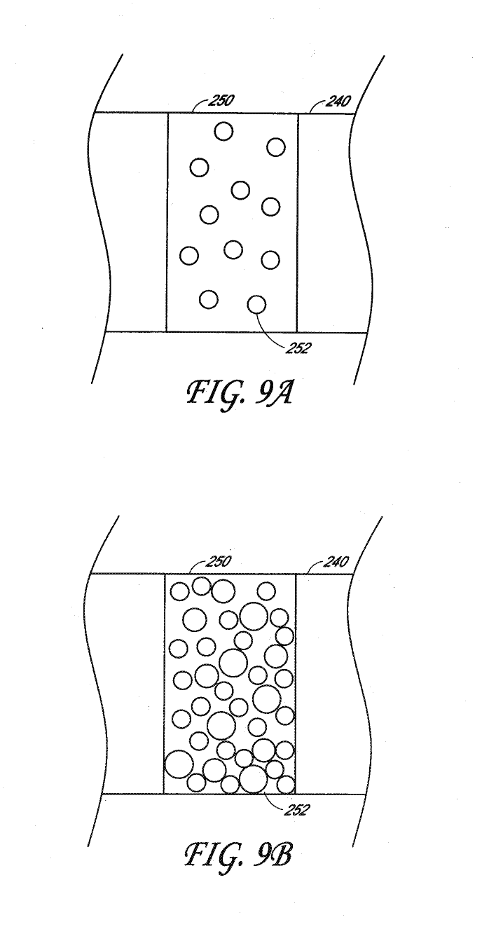

FIGS. 9A-9B show schematics of a partial embodiment of the capture region 250 in the release medium 240 before and after, respectively, application of an enhancement solution. While the capture region 250 is shown, it is understood that the enhancement region may also be any measurement region of the strip 200, such as the control region 260. In some embodiments, the enhancement solution may provide more of the label 252 at the capture region 250. In some embodiments, the label 252 is metal. For example, the label 252 may be gold, silver or other metals. In some embodiments, the label 252 may be a polymer. FIG. 9A shows the capture region 250 after application of a sandwich complex having the label 252 and before application of an enhancement solution. FIG. 9B shows the capture region 250 after application of a sandwich complex having the label 252 and after application of an enhancement solution. The enhancement solution may be applied in any of the manners discussed herein, for example by flowing one or more enhancement solutions from one or more stores after the fluid sample has flowed to the capture region 250. It is appreciated that the concentration of label 252 in FIG. 9B is greater than the concentration of the label 252 in FIG. 9A. Thus, the application of the enhancement solution may provide for more of the label 252 to be bound at the capture region 250. This in turn may make verification of the test results simpler, such as providing a stronger impedance measurement, darker visual band, etc.

Although this invention has been disclosed in the context of certain embodiments and examples, it will be understood by those skilled in the art that the present invention extends beyond the specifically disclosed embodiments to other alternative embodiments and/or uses of the invention and apparent modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

The logical blocks, modules and flow chart sequences are illustrative only. A person of skill in the art will understand that the steps, decisions, and processes embodied in the flowcharts described herein may be performed in an order other than that described herein. Thus, the particular flowcharts and descriptions are not intended to limit the associated processes to being performed in the specific order described.

Those of skill in the art will recognize that the various illustrative logical blocks, modules, and method steps described in connection with the embodiments disclosed herein may be implemented as electronic hardware, software stored on a computer readable medium and executable by a processor, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor reads information from, and writes information to, the storage medium. In the alternative, the storage medium may be integral to the processor. The processor and the storage medium may reside in an ASIC.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others. The scope of the invention is indicated by the appended claims rather than by the foregoing description. All changes which come within the meaning and range of equivalency of the claims are to be embraced within their scope.

A person skilled in the art will recognize that each of these sub-systems may be inter-connected and controllably connected using a variety of techniques and hardware and that the present disclosure is not limited to any specific method of connection or connection hardware.

The technology is operational with numerous other general purpose or special purpose computing system environments or configurations. Examples of well-known computing systems, environments, and/or configurations that may be suitable for use with the invention include, but are not limited to, personal computers, server computers, hand-held or laptop devices, multiprocessor systems, microprocessor-based systems, a microcontroller or microcontroller based system, programmable consumer electronics, network PCs, minicomputers, mainframe computers, distributed computing environments that include any of the above systems or devices, and the like.

As used herein, instructions refer to computer-implemented steps for processing information in the system. Instructions may be implemented in software, firmware or hardware and include any type of programmed step undertaken by components of the system.

The system may be used in connection with various operating systems such as Linux®, UNIX® or Microsoft Windows®.

The system control may be written in any conventional programming language such as C, C++, BASIC, Pascal, .NET (e.g., C#), or Java, and ran under a conventional operating system. C, C++, BASIC, Pascal, Java, and FORTRAN are industry standard programming languages for which many commercial compilers may be used to create executable code. The system control may also be written using interpreted languages such as Perl, Python or Ruby. Other languages may also be used such as PHP, JavaScript, and the like.

The foregoing description details certain embodiments of the systems, devices, and methods disclosed herein. It will be appreciated, however, that no matter how detailed the foregoing appears in text, the systems, devices, and methods may be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment may be included with other depicted embodiments in any combination. For example, any of the various components described herein and/or depicted in the Figures may be combined, interchanged or excluded from other embodiments.

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description discloses several methods, devices and systems of the present invention. This invention is susceptible to modifications in the methods, devices and systems. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the following claims.

What is claimed is:

1. A device for detecting an analyte in a fluid sample, the device comprising:
a test strip comprising:
a sample receiving region that receives the fluid sample; and
a capture region,
wherein the fluid sample flows laterally to the capture region upon receipt of the fluid sample by the sample receiving region; and
a first store located upstream of the capture region, the first store containing a first enhancement solution,
wherein the first store releases the first enhancement solution onto the strip after the sample receiving region receives the fluid sample, and
wherein, upon release onto the strip, the first enhancement solution flows laterally to the capture region.

2. The device of claim 1, further comprising a second store located upstream of the capture region, the second store containing a second enhancement solution wherein the second store releases the second enhancement solution onto the strip after the sample receiving region receives the fluid sample, and wherein, upon release onto the strip, the second enhancement solution flows laterally to the capture region.

3. The device of claim 2, wherein the first enhancement solution comprises a silver salt solution and the second enhancement solution comprises a hydroquinone initiator.

4. The device of claim 2, wherein the first enhancement solution comprises chloroauric acid (HAuCl₄) and the second enhancement solution comprises Hydroxylamine hydrochloride (NH₂OH.HCl).

5. The device of claim 1, wherein the first store further comprises a release, wherein actuating the release allows the first enhancement solution to exit the first store and flow toward the strip.

6. The device of claim 1, further comprising a wash store containing a washing fluid wherein the wash store releases the washing fluid onto the strip after the sample receiving region receives the fluid sample, and wherein, upon release onto the strip, the washing fluid flows laterally to the capture region.

7. The device of claim 6, wherein the wash store further comprises a wash release, wherein actuating the wash release allows the washing fluid to exit the wash store and flow toward the strip.

8. The device of claim 5, further comprising an electronic circuit comprising a processor operatively coupled to the release, the processor configured to execute a set of instructions to perform a method comprising actuating the release.

9. The device of claim 7, further comprising an electronic circuit comprising a processor operatively coupled to the wash release, the processor configured to execute a set of instructions to perform a method comprising actuating the wash release.

10. The device of claim 8, further comprising a plurality of electrodes at least partially aligned with the capture region, wherein the processor is further coupled to the electrodes, and wherein the method further comprises measuring an electrical property of the capture region.

11. The device of claim 2, wherein the first store comprises a first release and the second store comprises a second release, wherein actuating the first release causes the first enhancement solution to exit the first store and flow toward the strip, and wherein actuating the second release causes the second enhancement solution to exit the second store and flow toward the strip.

12. The device of claim 1, the strip further comprising:
a first antibody region comprising a first antibody that recognizes an epitope of the analyte; and
a second antibody region comprising a second antibody that recognizes a different epitope of the analyte.

13. The device of claim 12, wherein the first antibody is bound to a first label, and wherein the second antibody is bound to a second label.

14. The device of claim 1, the strip further comprising a release medium, a capture medium, an absorbent medium and a backing.

15. The device of claim 14, wherein the release medium comprises:
the sample receiving region;
a first antibody region comprising a first antibody that recognizes an epitope of the analyte; and
a second antibody region comprising a second antibody that recognizes a different epitope of the analyte.

16. A method for detecting an analyte in a fluid sample, the method comprising:
applying a fluid sample to a strip, wherein the strip is configured such that the fluid sample flows laterally to a capture region of the strip; and
releasing a first enhancement solution from a first store coupled with the strip, wherein the first enhancement solution flows from the first store and onto the strip after the sample receiving region receives the fluid sample, and wherein, upon flowing onto the strip, the first enhancement solution flows laterally to the capture region.

17. The method of claim 16, further comprising releasing a second enhancement solution from a second store coupled with the strip, wherein the second enhancement solution flows from the second store and onto the strip after the sample receiving region receives the fluid sample, and wherein, upon flowing onto the strip, the second enhancement solution flows laterally to the capture region.

18. The method of claim 17, wherein the first enhancement solution comprises a silver salt solution and the second enhancement solution comprises a hydroquinone initiator.

19. The method of claim 17, wherein the first enhancement solution comprises chloroauric acid (HAuCl₄) and the second enhancement solution comprises Hydroxylamine hydrochloride (NH₂OH.HCl).

20. The method of claim 16, wherein releasing the first enhancement solution comprises actuating a first release to allow the first enhancement solution to flow from the first store.

21. The method of claim 20, wherein actuating the first release comprises mechanically pushing the first enhancement solution from the first store and toward the strip.

22. The method of claim 20, wherein actuating the first release comprises puncturing the first store containing the first enhancement solution to allow the first enhancement solution to exit the first store and flow toward the strip.

23. The method of claim 17, wherein releasing the first and second enhancement solutions comprises, respectively, actuating a first and second release to allow, respectively, the first and second enhancement solutions to flow from the first and second stores.

24. The method of claim 23, further comprising measuring an electrical property of the capture region, wherein the first and second release are actuated after the measured electrical property of the capture region reaches a threshold value.
